(19) United States
(12) Patent Application Publication
(10) Pub. No.: US 2026/0191560 A1
(43) Pub. Date: Jul. 9, 2026

(54) ROBOTIC NAVIGATION ASSISTED SURGICAL TOOL GUIDANCE

(71) Applicant: Alphatec Spine, Inc., Carlsbad, CA (US)

(72) Inventors: Biko Hartmann, Louisville, CO (US); Lucas Hoffmann, Denver, CO (US)

(21) Appl. No.: 18/868,337

(22) PCT Filed: May 24, 2023
(86) PCT No.: PCT/US2023/023369

§ 371 Date: Nov. 22, 2024

Related U.S. Application Data
(60) Provisional application No. 63/345,340, filed on May 24, 2022.

(51) Int. Cl. A61B 17/34; A61B 17/3211; A61B 34/20; A61B 34/30

(52) U.S. Cl. CPC A61B 17/3421; A61B 17/3211; A61B 34/20; A61B 34/30; A61B 2034/2055; A61B 2034/303

(57) ABSTRACT

A spinal surgical system includes a tissue protector with an elongate body, and a dilator sized and shaped to be removably inserted into the lumen of the tissue protector. The dilator includes a connector for engaging a tracking array of a robotic navigation system, and the tissue protector is also engageable with a robot of the robotic navigation system. For example, the robot may have a guide tube and the tissue protector can be inserted into the guide tube. The tracking array can include a body with a plurality of arms, with tracker(s) connectable to the plurality of arms.

### RELATED APPLICATIONS

This application claims priority to U.S. provisional application 63/345,340, filed 24 May 2022, the entire contents of which are incorporated herein by reference.

### FIELD OF THE DISCLOSURE

The present disclosure relates to specialized tools for spinal surgery, including guidance instruments, and surgical methods for using the same.

### SUMMARY

The present disclosure relates to a set of guidance instruments for use in surgical procedures, in particular, surgical spinal procedures. The device includes a tapered cannula having a tissue contacting distal end, a tissue tool insertion proximal end, and a slotted through channel from the proximal to the distal end. The device can also include a tissue dilator insertable through the tapered cannula, with the dilator having a shaped distal end for separating and penetrating tissue and—in some configurations—engaging and/or penetrating a target bony structure, and near a proximal end having a circumferential receiver. The device can further include a tracking array that includes one or a plurality of navigation trackers, a housing having a through channel for receiving a proximal portion of the tissue dilator, and one or more engagement tabs for engaging the circumferential receiver on the tissue dilator to retain the tracking array to the tissue dilator.

A spinal surgical system can include: (i) a tissue protector comprising an elongate body having a proximal end, a distal end, and a lumen extending therethrough from the proximal end to the distal end; (ii) a dilator sized and shaped to be removably insertable into the lumen of the tissue protector, the dilator comprising an elongate body extending from a proximal end for separating tissue and a distal end; and (iii) a tracking array of a robotic navigation system selectively connectable to the dilator. In some configurations, the tissue protector is engageable with a robot of the robotic navigation system.

According to one aspect, the tracking array of the robotic navigation system can be visualized by a camera of the robotic navigation system, allowing optical tracking and navigation of the dilator within the tissue protector and through soft tissue. The robotic navigation system may provide real-time visualization and navigation of the dilator and tissue protector within soft tissue.

According to one aspect, the robot of the robotic navigation system comprises a guide tube, and the tissue protector and/or other instruments may be insertable into the guide tube.

The dilator may include a connector for engaging the tracking array, the connector positioned toward the distal end of the dilator. In other configurations, the dilator does not include a connector and instead the tracking array includes engagement means for selectively engaging the dilator.

In some configurations, the tissue protector comprises a plurality of relief channels formed on an external surface of the elongate body to provide improved movement when traversing through soft tissue. In one configuration, the relief channels are longitudinally arranged around the elongate body of the tissue protector. In one configuration, the relief channels are arranged in a helical manner around the elongate body of the tissue protector. The tissue protector may have a distal end for engaging bone, such as a toothed distal end. The tissue protector can be provided in multiple sizes, including different lengths and circumferences.

In one aspect, the tissue protector can be provided with a single outer diameter and multiple inner diameters and/or multiple lengths. In one aspect, the tissue protector can be provided in multiple outer diameters with multiple or a single inner diameter and/or multiple or a single length.

According to another aspect, the dilator may have a distal end for engaging bone. In yet another aspect, the distal end is configured to penetrate bone in addition to or as an alternative to just engaging the bone. In some configurations, the dilator has a distal end for traversing through soft tissue.

According to another aspect, the tracking array can include a tracking array body removably attachable to the dilator, a plurality of arms extending outwardly from the tracking array body, with each of the plurality of arms having a distal end, and a plurality of trackers, the trackers attachable to the distal end of one of the plurality of arms. The trackers are optically detectable by the robotic navigation system to facilitate navigation of the dilator during spinal surgery.

The tracking array body can include an attachment mechanism to securely attach the tracking array body to the dilator. In one configuration, the tracking array body comprises a through-channel for selectively receiving a portion of the dilator.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be better understood when read in conjunction with the following drawings wherein like structure is indicated with like reference numerals and in which:

FIG. 1 is a perspective view of a tissue protector-dilator assembly;

FIG. 2 is a cross-sectional view of the configuration shown in FIG. 1;

FIG. 3 is a perspective view of another configuration of a tissue protector;

FIG. 4 is a cross-sectional view of the configuration of a tissue protector shown in FIG. 3;

FIG. 5 is a perspective view of one configuration of a dilator;

FIGS. 6-8 are perspective views of configurations of distal ends of a dilator;

FIG. 9 is a perspective view of a configuration of a tracking array;

FIG. 10 is another perspective view of the tracking array of FIG. 9;

FIG. 11 is a perspective view of a tracking array attached to a tissue protector-dilator assembly, with the assembly inserted into the guide tube of a robot of a robotic navigation system;

FIG. 12 is a perspective view of a scalpel handle; and

FIG. 13 is an exemplary method that can be used to form a surgical channel with the systems described herein.

### DETAILED DESCRIPTION

The present disclosure relates to a system that can be used to create a working surgical channel for surgeons during spinal surgery. The channel may allow surgeons to use navigated and/or non-navigated instruments in conjunction with a robotic navigation system. In one aspect, the system includes a tissue protector with a hollow lumen, and a dilator that is insertable through the hollow lumen. The tissue protector may be insertable through a guide tube of a surgical robot. The system can also include a scalpel and/or scalpel handle that is insertable through the guide tube of the surgical robot to create an incision. The system can also include one or more tracking arrays attachable to the dilator.

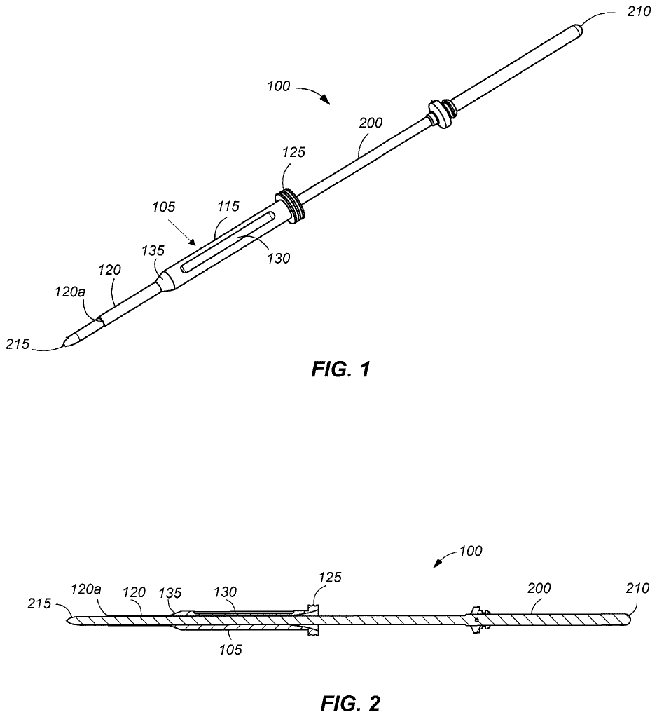

FIGS. 1-2 illustrate one embodiment of a tissue protector-dilator assembly 100. FIG. 1 shows the tissue protector-dilator assembly 100 includes a cannula or tissue protector 105, and a dilator 200 which is insertable into a lumen 110 (visible in FIG. 4) of the tissue protector 105. The dilator 200 is movable relative to the tissue protector 105 along the longitudinal axis of the tissue protector 105 and/or the dilator 200.

The tissue protector 105 includes an elongate body 115 extending from a distal end 120 to a proximal end 125. The distal end 120 (including distal edge 120a) is inserted into an incision, and the proximal end 125 is typically where tools (such as the dilator 200, and/or other tools) are inserted into the lumen 110. The elongate body 115 defines the lumen 110.

The tissue protector 105 can be provided in multiple sizes, including multiple diameters and/or multiple lengths to accommodate various instruments and needs of the patient. The tissue protector 105 can be engageable with a robot of a robotic navigation system. For example, a robot can include a guide tube for selectively receiving the tissue protector 105 as described in more detail below.

The tissue protector 105 can also be provided with relief channels 130 to improve movement of the tissue protector 105 through soft tissue. Relief channels 130 may be formed on an external surface 135 of the elongate body 115 to provide improved movement when traversing through soft tissue. Relief channels 130 can be formed in any shape or size desired. In some configurations, relief channels 130 are longitudinally arranged around the elongate body 115 (e.g., around the circumference) of the tissue protector 105. In some configurations, relief channels 130 may form a helical pattern along the exterior of the elongate body 115.

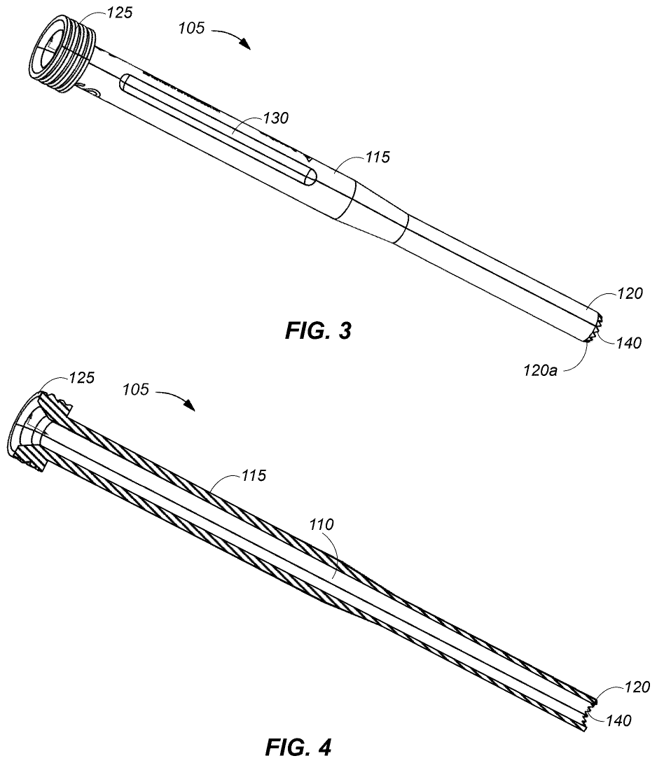

Various configurations for the distal end 120 and distal edge 120a of the distal end 120 of the tissue protector 105 can be provided, for example, to improve bone engagement. For example, FIGS. 3-4 illustrate a configuration with a toothed distal end 120. When the tissue protector 105 is fully inserted into an incision to bone, the teeth 140 of the distal end 120, which project outwardly from the distal edge 120a, can engage bone and reduce slippage of the distal end 120 relative to the bone. In some configurations, the proximal end 125 can be tapped with a driver or other means to more fully engage the teeth 140 with the bone. In other configurations, the tissue protector 105 can have a smooth distal tip 120a to improve soft tissue dilation.

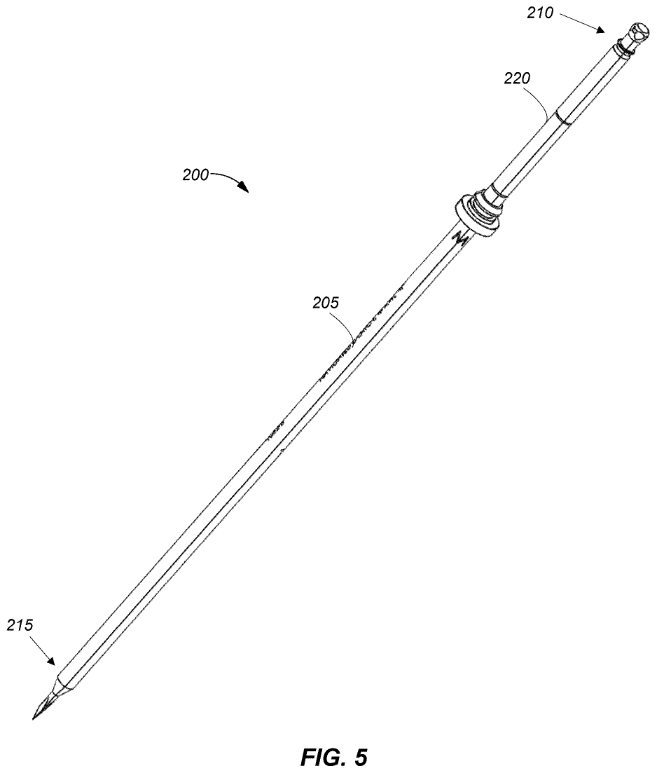

FIG. 5 illustrates a configuration of a dilator 200 that can be used with the tissue protector 105. The dilator 200 is sized and shaped to be removably insertable into the lumen 110 of the tissue protector 105 (as shown in FIGS. 1-2). The dilator 200 generally comprises an elongate body 205 extending from a proximal end 210 to a distal end 215 for separating tissue. The dilator 200 is also connectable to a tracking element of a robotic navigation system. In some configurations, the dilator 200 includes a connector 220 or other engagement means for engaging the tracking element. In other configurations, the tracking element is directly attachable to the dilator 200 without the dilator 200 having any engagement means. Connector 220 may be, for example, a slot or indent formed near the distal end 215 of the dilator 200.

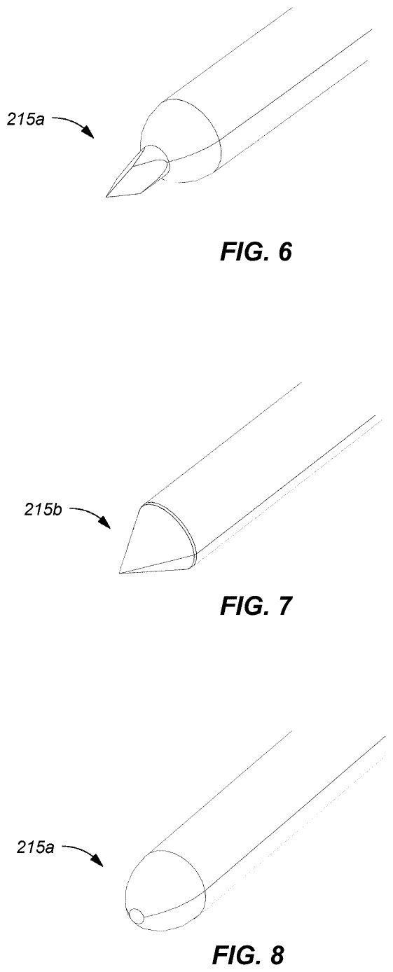

Like the tissue protector 105, the dilator 200 can be configured with various distal end 215 configurations in addition to the design illustrated in FIG. 5. FIGS. 6-8 are given by way of illustration, and not limitation, for various distal end 215 configurations. Depending on the desired results, a surgeon may use one or more dilators 200 with different distal ends 215 to achieve the desired surgical channel. For example, tips such as those shown in FIG. 5 (215, an awl tip) and FIG. 6 (215a, a bullet-awl tip) may improve bone engagement and/or penetration, whereas a blunt tip such as the conical tip 215b shown in FIG. 7 and the blunt tip 215c shown in FIG. 8 may improve soft tissue dilation.

The dilator 200 also includes connector 220 or another suitable engagement means for engaging a tracking element of a robotic or surgical tracking system. The tracking element can be any suitable tracking element that is compatible with the robotic system, such as a radiofrequency coil, a magnetic tracking element, an optical tracking element, and/or an ultrasonic tracking element. In one configuration, the robotic tracking system includes a camera and the tracking element connectable to the dilator is an optical tracking element such as a tracking array 300.

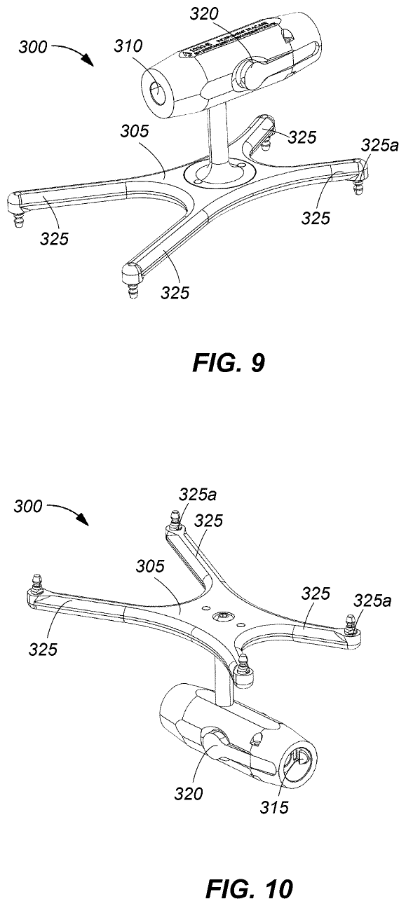

FIGS. 9-10 show perspective views of one configuration of a tracking array 300. The tracking array 300 may be any suitable shape and size to be selectively attached to the dilator 200 and tracked by a camera of the robotic navigation system. For example, the tracking array 300 may include a body 305 with a through-channel 310 to receive a distal portion 215 of the dilator 200. The connector 220 of the dilator 200 can engage the through-channel 310 of the tracking array 300 to secure the dilator 200 to the tracking array 300.

In some configurations, the tracking array 300 can also be provided with means to engage a connector 220 of the dilator 200. For example, the tracking-array 300 may include one or more tabs 315 to engage the connector 220 (e.g., slot/indent) of the dilator 200. Tab(s) 315 can be biased inwardly, such as via a spring. Pressing actuator(s) 320 can move the tab(s) 315 outwardly as the through-channel 310 is moved along the elongate body 205 of the tissue dilator 200. Then, when the tracking array 300 reaches the desired position, the actuator(s) 320 can be released and the tab(s) 315 returned to their inwardly biased position. Tab(s) 315 can engage connector 220 of the dilator 200. Other engagement means (either active or passive) are also contemplated.

The body 305 of the tracking array 300 can include one or more arms 325 extending outwardly from the body 305. Arm(s) 325 may have a distal end 325a for engaging trackers of any shape or size desired (trackers not shown in FIGS. 9-10). Four arms 325 may be used, or fewer or more arms may be used as desired. The trackers may be removably or non-removably attached to the distal end 325a of the arms 325. The trackers may be any suitable tracking element that is compatible with the robotic system, such as a radiofrequency coil, a magnetic tracking element, an optical tracking element, and/or an ultrasonic tracking element. In one configuration, the robotic navigation system includes a camera and the trackers are optical trackers. For example, the trackers can be optically detectable by the robotic navigation system to facilitate navigation of the dilator 200 during spinal surgery.

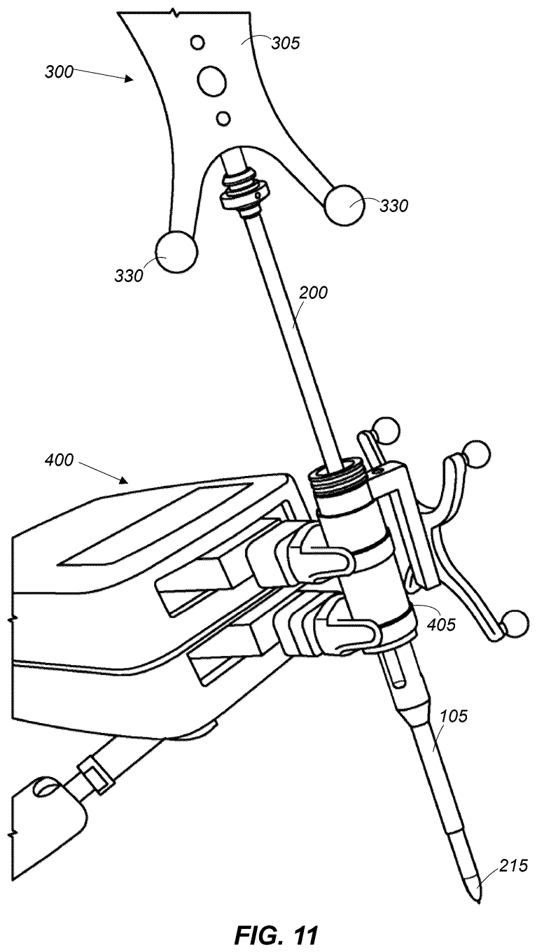

FIG. 11 shows an assembled tissue protector-dilator assembly 100 with a tracking array 300 connected to the dilator 200. The tissue protector 105 of the assembled system 100 is shown inserted into a guide tube 405 of a robot 400 of a robotic navigation system. The robotic navigation system may also include one or more cameras (not illustrated). The tracking array 300 shown in FIG. 11 includes a body 305 with four outwardly extending arms 325, forming a generally rectangular shape, with substantially spherical trackers 330. These trackers 330 can allow visualization and navigation with a robotic navigation system.

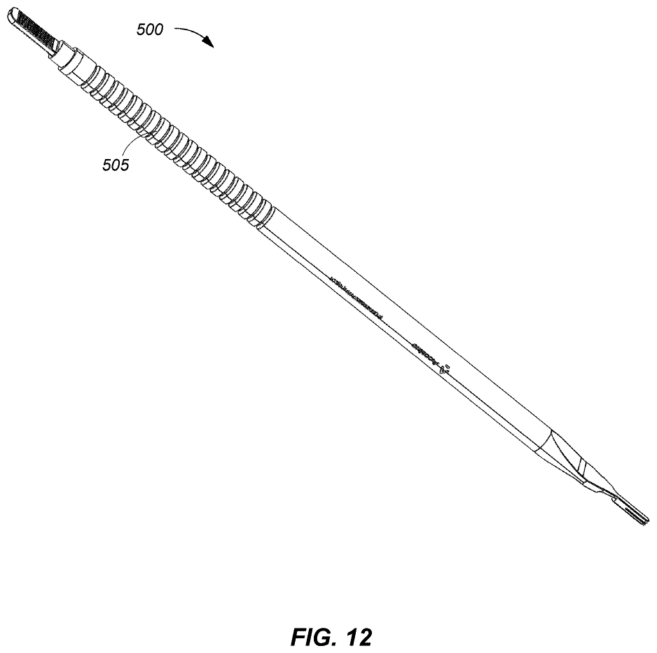

In some configurations, the system can also include other surgical tools that are compatible with the robot 400, such as tools that fit within the robot's guide tube 405. For example, a scalpel handle 500 can be provided (FIG. 12). Like the tissue protector 105, the scalpel handle 500 is engageable with a robot 400 of a robotic navigation system. In some configurations, the robot 400 includes a guide tube 405, and the scalpel handle 500 is insertable into the guide tube 405 of the robot 400. The scalpel handle 500 can be compatible with any type of suitable scalpel blade, and may include an ergonomic ribbed handle 505. By fitting through the guide tube 405 of the robot 400, the scalpel handle 500 can align with a planned trajectory for the surgical channel.

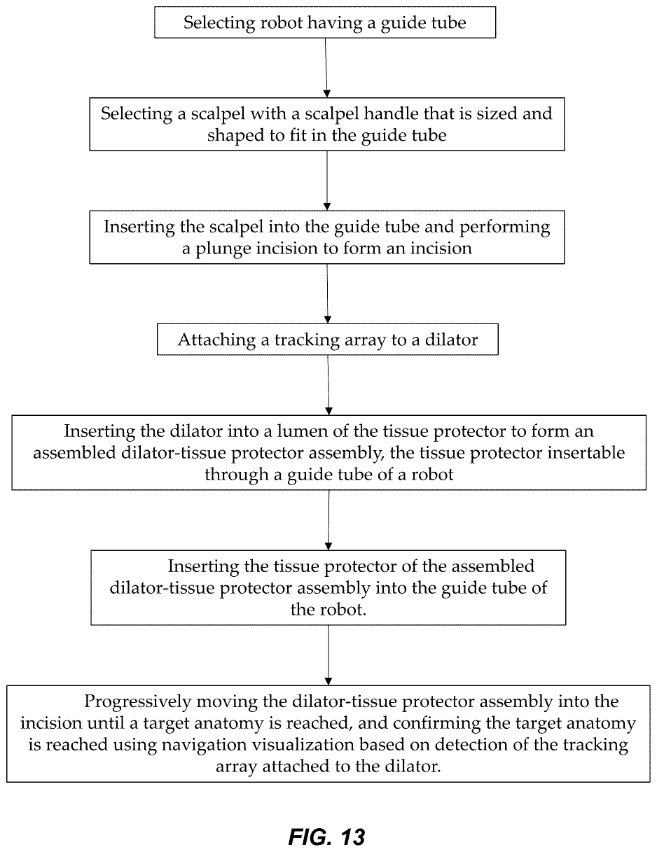

In use, the assembled dilator-tissue protector system 100 can be inserted into a patient to create a working channel to prepare a pedicle of the spine for surgery. The working channel protects tissue, and navigable and non-navigable instruments can be used in the channel. FIG. 13 shows a flow chart of a method that can be used with the instruments described herein.

First, a plunge incision can be performed. Any suitable scalpel can be used to perform the plunge incision. For example, a scalpel with a scalpel handle 500 that can fit through the guide tube 405 of the robot 400 can be used. In some aspects, the system can include a scalpel handle that is sized and shaped to pass through the guide tube of the robot. Any desired disposable scalpel blade can be attached to the handle of the scalpel. After attaching the preferred standard disposable scalpel blade to the scalpel handle, the surgeon can perform a plunge incision.

Next, the dilator-tissue protector assembly 100 may be formed. A tracking array is attached to the distal portion of the dilator, and the dilator is inserted into the lumen of the tissue protector. This assembly can then be inserted into the guide tube of the robot by inserting the tissue protector of the assembled dilator-tissue protector assembly into the guide tube of the robot.

The assembly can then be inserted into the incision and progressively moved deeper into the incision until a target anatomy is reached. As the assembly moves deeper into the incision, movement of the assembly is tracked and visualized on-screen using the robotic navigation system (which includes a camera for detecting and tracking the trackers of the tracking array). After the assembly reaches the desired position, the dilator can be removed from the tissue protector to expose the working channel that has been formed. Subsequently, navigable and non-navigable instruments may be inserted into the working channel, for example, to prepare a pedicle. Such preparation may include preparation for the placement of pedicle screws to serve as a part of a posterior fixation construct.

### EMBODIMENTS

The following embodiments are provided as examples only of specific configurations, materials, arrangements, etc. contemplated by the authors of this disclosure:

Embodiment 1: A spinal surgical system comprising: a tissue protector comprising an elongate body having a proximal end, a distal end, and a lumen extending therethrough from the proximal end to the distal end; a dilator sized and shaped to be removably insertable into the lumen of the tissue protector, the dilator comprising an elongate body extending from a proximal end for separating tissue and a distal end; and the dilator selectively engageable with a tracking array of a robotic navigation system and wherein the tissue protector is engageable with a robot of the robotic navigation system.

Embodiment 2: The spinal surgical system of Embodiment 1, wherein the tracking array of the robotic navigation system can be visualized by a camera of the robotic navigation system, allowing optical tracking and navigation of the dilator within the tissue protector and through soft tissue.

Embodiment 3: The spinal surgical system of Embodiment 1 or 2, wherein the dilator comprises a connector for engaging the tracking array, the connector positioned toward the distal end of the dilator.

Embodiment 4: The spinal surgical system of any one of Embodiments 1-3, wherein the tissue protector comprises a plurality of relief channels formed on an external surface of the elongate body to provide improved movement when traversing through soft tissue.

Embodiment 5: The spinal surgical system of Embodiment 4, wherein the relief channels are longitudinally arranged around the elongate body of the tissue protector.

Embodiment 6: The spinal surgical system of any one of Embodiments 1-5, wherein the robotic navigation system provides real-time visualization and navigation of the dilator and tissue protector within soft tissue.

Embodiment 7: The spinal surgical system of any one of Embodiments 1-6, wherein the robot of the robotic navigation system comprises a guide tube and wherein the tissue protector is insertable into the guide tube.

Embodiment 8: The spinal surgical system of any one of Embodiments 1-7, wherein the tissue protector has a distal end for engaging bone.

Embodiment 9: The spinal surgical system of Embodiment 8, wherein the tissue protector has a toothed distal end.

Embodiment 10: The spinal surgical system of any one of Embodiments 1-9, wherein the tissue protector may be provided in various lengths and/or widths to accommodate surgical instruments and/or patient needs.

Embodiment 11: The spinal surgical system of any one of Embodiments 1-10, wherein the dilator has a distal end for engaging bone.

Embodiment 12: The spinal surgical system any one of Embodiments 1-11, wherein the dilator has a distal end for traversing through soft tissue.

Embodiment 13: The spinal surgical system of any one of Embodiments 1-12, wherein the tracking array comprises a tracking array body removably attachable to the dilator, a plurality of arms extending outwardly from the tracking array body, each of the plurality of arms having a distal end, and a plurality of trackers, the trackers attachable to the distal end of one of the plurality of arms.

Embodiment 14: The spinal surgical system of Embodiment 13, wherein the trackers are optically detectable by the robotic navigation system to facilitate navigation of the dilator during spinal surgery.

Embodiment 15: The spinal surgical system of Embodiment 13, wherein the tracking array body comprises an attachment mechanism to securely attach the tracking array body to the dilator.

Embodiment 16: The spinal surgical system of Embodiment 15, wherein the tracking array body comprises a through-channel for selectively receiving a portion of the dilator.

Embodiment 17: A spinal surgical system comprising: a tissue protector comprising an elongate body having a proximal end, a distal end, and a lumen extending therethrough from the proximal end to the distal end, the tissue protector insertable through a guide tube of a robot; a dilator sized and shaped to be removably insertable into the lumen of the tissue protector, the dilator comprising an elongate body extending from a proximal end for passing through tissue and a distal end; a tracking array selectively connectable to the dilator; and a scalpel handle insertable through the guide tube of the robot.

Embodiment 18: The spinal surgical system of Embodiment 17, wherein the tracking array can be visualized by a camera of a robotic navigation system, allowing optical tracking and navigation of the dilator within the tissue protector and through soft tissue.

Embodiment 19: A spinal surgical system comprising: a tissue protector comprising an elongate body having a proximal end, a distal end, and a lumen extending therethrough from the proximal end to the distal end; a dilator sized and shaped to be removably insertable into the lumen of the tissue protector, the dilator comprising an elongate body extending from a proximal end for separating tissue and a distal end; and the dilator selectively engageable with a tracking array of a robotic navigation system.

Embodiment 20: A method of forming a working channel for surgery, the method comprising: selecting a robot having a guide tube; selecting a scalpel with a scalpel handle that is sized and shaped to fit in the guide tube; inserting the scalpel into the guide tube and performing a plunge incision to form an incision; attaching a tracking array to a dilator, the dilator sized and shaped to be removably insertable into a lumen of a tissue protector, the dilator comprising an elongate body extending from a proximal end for passing through tissue and a distal end; inserting the dilator into the lumen of the tissue protector to form an assembled dilator-tissue protector assembly, the tissue protector comprising an elongate body having a proximal end, a distal end, and the lumen extending therethrough from the proximal end to the distal end, the tissue protector insertable through the guide tube of the robot; and inserting the tissue protector of the assembled dilator-tissue protector assembly into the guide tube of the robot.

Embodiment 21: The method of Embodiment 20, further comprising progressively moving the dilator-tissue protector assembly into the incision until a target anatomy is reached, and confirming the target anatomy is reached using navigation visualization based on detection of the tracking array attached to the dilator.

Embodiment 22: The method of Embodiment 21, further comprising removing the dilator from the tissue protector and maintaining the tissue protector in place relative to the guide tube to allow one or more instruments to access the target anatomy through the tissue protector.

Embodiment 23: A spinal surgical kit comprising: a tissue protector comprising an elongate body having a proximal end, a distal end, and a lumen extending therethrough from the proximal end to the distal end; a dilator sized and shaped to be removably insertable into the lumen of the tissue protector, the dilator comprising an elongate body extending from a proximal end for separating tissue and a distal end; and the dilator selectively engageable with a tracking array of a robotic navigation system.

Embodiment 24: The spinal surgical kit of Embodiment 24, wherein the tissue protector is provided in a first length and a second length.

Embodiment 25: The spinal surgical kit of Embodiment 24, wherein the dilator is provided in a first configuration with a rounded distal end and a second configuration with a pointed distal end.

Embodiment 26: The spinal surgical kit of Embodiment 24, further comprising a scalpel handle sized and shaped to be selectively engageable with a guide tube of a robot of a robotic navigation system.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination.

Unless otherwise indicated, all numbers expressing measurements used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the embodiments of the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. In one embodiment, the terms “about” and “approximately” refer to numerical parameters within 10% of the indicated range.

The terms “a,” “an,” “the,” and similar referents used in the context of describing the embodiments of the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the embodiments of the present disclosure and does not pose a limitation on the scope of the present disclosure. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the author(s) of this disclosure for carrying out the embodiments disclosed herein. Of course, variations on these described embodiments will become apparent to those of ordinary skill in the art upon reading the foregoing description. The author(s) expects skilled artisans to employ such variations as appropriate, and the author(s) intends for the embodiments of the present disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of this disclosure so claimed are inherently or expressly described and enabled herein.

Furthermore, if any references have been made to patents and printed publications throughout this disclosure, each of these references and printed publications are individually incorporated herein by reference in their entirety.

The embodiments disclosed herein are illustrative of the principles of the present disclosure. Other modifications that may be employed are within the scope of this disclosure. Thus, by way of example, but not of limitation, alternative configurations of the embodiments of the present disclosure may be utilized in accordance with the teachings herein. Accordingly, the present disclosure is not limited to that precisely as shown and described.

1. A spinal surgical system comprising:
a tissue protector comprising an elongate body having a proximal end, a distal end, and a lumen extending therethrough from the proximal end to the distal end;
a dilator sized and shaped to be removably insertable into the lumen of the tissue protector, the dilator comprising an elongate body extending from a proximal end for separating tissue and a distal end; and
the dilator selectively engageable with a tracking array of a robotic navigation system and wherein the tissue protector is engageable with a robot of the robotic navigation system.

2. The spinal surgical system of claim 1, wherein the tracking array of the robotic navigation system can be visualized by a camera of the robotic navigation system allowing optical tracking and navigation of the dilator within the tissue protector and through soft tissue.

3. The spinal surgical system of claim 1, wherein the dilator comprises a connector for engaging the tracking array, the connector positioned toward the distal end of the dilator.

4. The spinal surgical system of claim 1, wherein the tissue protector comprises a plurality of relief channels formed on an external surface of the elongate body to provide improved movement when traversing through soft tissue.

5. The spinal surgical system of claim 4, wherein the relief channels are longitudinally arranged around the elongate body of the tissue protector.

6. The spinal surgical system of claim 1, wherein the robotic navigation system provides real-time visualization and navigation of the dilator and tissue protector within soft tissue.

7. The spinal surgical system of claim 1, wherein the robot of the robotic navigation system comprises a guide tube and wherein the tissue protector is insertable into the guide tube.

8. The spinal surgical system of claim 1, wherein the tissue protector has a distal end for engaging bone.

9. The spinal surgical system of claim 8, wherein the tissue protector has a toothed distal end.

10. The spinal surgical system of claim 1, wherein the tissue protector may be provided in various lengths and/or widths to accommodate surgical instruments and/or patient needs.

11. The spinal surgical system of claim 1, wherein the dilator has a distal end for engaging bone.

12. The spinal surgical system of claim 1, wherein the dilator has a distal end for traversing through soft tissue.

13. The spinal surgical system of claim 1, wherein the tracking array comprises a tracking array body removably attachable to the dilator, a plurality of arms extending outwardly from the tracking array body, each of the plurality of arms having a distal end, and a plurality of trackers, the trackers attachable to the distal end of one of the plurality of arms.

14. The spinal surgical system of claim 13, wherein the trackers are optically detectable by the robotic navigation system to facilitate navigation of the dilator during spinal surgery.

15. The spinal surgical system of claim 13, wherein the tracking array body comprises an attachment mechanism to securely attach the tracking array body to the dilator.

16. The spinal surgical system of claim 15, wherein the tracking array body comprises a through-channel for selectively receiving a portion of the dilator.

17. A spinal surgical system comprising:
a tissue protector comprising an elongate body having a proximal end, a distal end, and a lumen extending therethrough from the proximal end to the distal end, the tissue protector insertable through a guide tube of a robot;
a dilator sized and shaped to be removably insertable into the lumen of the tissue protector, the dilator comprising an elongate body extending from a proximal end for passing through tissue and a distal end;
a tracking array selectively connectable to the dilator; and
a scalpel handle insertable through the guide tube of the robot.

18. The spinal surgical system of claim 17, wherein the tracking array can be visualized by a camera of a robotic navigation system, allowing optical tracking and navigation of the dilator within the tissue protector and through soft tissue.

19. A spinal surgical system comprising:
a tissue protector comprising an elongate body having a proximal end, a distal end, and a lumen extending therethrough from the proximal end to the distal end;
a dilator sized and shaped to be removably insertable into the lumen of the tissue protector, the dilator comprising an elongate body extending from a proximal end for separating tissue and a distal end; and
the dilator selectively engageable with a tracking array of a robotic navigation system.

20. A method of forming a working channel for surgery, the method comprising:
selecting a robot having a guide tube;
selecting a scalpel with a scalpel handle that is sized and shaped to fit in the guide tube; inserting the scalpel into the guide tube and performing a plunge incision to form an incision;
attaching a tracking array to a dilator, the dilator sized and shaped to be removably insertable into a lumen of a tissue protector, the dilator comprising an elongate body extending from a proximal end for passing through tissue and a distal end;
inserting the dilator into the lumen of the tissue protector to form an assembled dilator-tissue protector assembly, the tissue protector comprising an elongate body having a proximal end, a distal end, and the lumen extending therethrough from the proximal end to the distal end, the tissue protector insertable through the guide tube of the robot; and
inserting the tissue protector of the assembled dilator-tissue protector assembly into the guide tube of the robot.

21. (canceled)

22. (canceled)
